(19) United States
(12) Patent Application Publication
(10) Pub. No.: US 2018/0043145 A1
(43) Pub. Date: Feb. 15, 2018

(54) APPLICATOR

(71) Applicant: MEDICAL BRANDS RESEARCH B.V., Amsterdam (NL)

(72) Inventors: Maikel HENDRIKS, Amsterdam (NL); Pieternella Anna Maria BOUTER, Amsterdam (NL)

(21) Appl. No.: 15/555,651

(22) PCT Filed: Mar. 4, 2016
(86) PCT No.: PCT/EP2016/054697

§ 371 Date: Sep. 5, 2017

(30) Foreign Application Priority Data
Mar. 6, 2015 (EP) 15158107.1

(51) Int. Cl. A61M 35/00; A61K 9/00; A61Q 19/00; A61Q 19/08; A61K 31/19; A61K 8/36

(52) U.S. Cl. CPC A61M 35/003; A61K 31/19; A61K 8/36; A61Q 19/00; A61Q 19/08; A61K 9/0014; A61K 2800/872

(57) ABSTRACT

Applicator (1) for topical application of an acidic liquid (30), the applicator comprising a container (3) containing said acidic liquid, the container comprising an opening (31) in fluid contact with a nib (2) for a controlled release of the acidic liquid, wherein the nib comprises a plurality of fibers (20) and an adhesive material (21) adhering the fibers. A kit of parts (100) for topical application of an acidic liquid, the kit of parts comprising (a) a liquid release unit (40) comprising a nib for a controlled release of the acidic liquid, and (b) a container unit (50) comprising a container configured to contain the acidic liquid, wherein the container unit further comprises a liquid release element for application of the acidic liquid to the nib, wherein the nib comprises a plurality of fibers and an adhesive material adhering the fibers.

### FIELD OF THE INVENTION

The invention relates to an applicator as well as a kit of parts for topical application of a liquid, such as for instance for treatment of a topical lesion, as well as to the liquid per se.

### BACKGROUND OF THE INVENTION

Various compositions are known for the treatment of skin lesions, such as warts, corn and calluses, actinic keratosis, keratosis pilaris, acne, skin hyperpigmentation and/or nail lesions, such as ingrown toenails and/or lesions in mucous membranes, such as cold sores and mouth ulcers. WO2012007584, for instance, describes a composition for the treatment of superficial lesions, in particular skin lesions, mucous membrane lesions and/or nail lesions, an applicator comprising such a composition and the use of such a composition. The composition comprises an effective amount of trichloroacetic acid, at least one thickener, and at a physiologically acceptable solvent. The composition is considered effective against a plethora of superficial lesions selected from the group consisting of viral warts, verrucae, water warts (molluscum contagiosum), corns and calluses, and skin hyperpigmentation: age spots, solar lentigo, senial lentigo, acne, keratosis pilaris, actinic keratosis, mouth ulcers (canker sores), cold sores, ingrown toenails, onychomycosis, eyelid xanthelasma.

US2011/245784 describes compositions, methods and systems for treating disordered epithelial tissues, such as is caused by pathogens and/or by toxins produced thereby. The invention relates to the use of an anti-infective and/or antimicrobial active agent in a carrier, with vigorous agitation of the disordered epithelial tissue for topical treatment thereof under such conditions sufficient to achieve clinically discernable improvement of the disordered epithelial tissue. The preferred anti-infective and/or antimicrobial active agent comprises an organohalide, such as a quaternary ammonium halide compound, preferably benzalkonium chloride. The inventive compositions and methods may employ the use of an applicator adapted for use in promoting the penetration of the treatment composition and/or the vigorous agitation of the disordered tissue.

EP2730310 describes a solution applicator with which irritation on an affected part of a patient may be reduced even when a solution is used. According to the present invention, an applicator comprises a solution container which comprises an opening, and a columnar brush member formed by bundling synthetic fibers in a columnar shape. The columnar brush member is disposed at the opening of the solution container, a tip portion of the columnar brush member at an outside of the solution container has a fan shape expanding in a perpendicular lateral direction against a pillar axial lengthwise direction, and a thickness of the fan-shaped tip portion of the columnar brush member decreases in a perpendicular lengthwise direction against the pillar axial lengthwise direction toward the tip portion of the columnar brush member. The applicator has the fan-shaped tip portion so that irritation on an affected part may be decreased and a liquid tinea unguium medicine may be applied to the affected part.

U.S. Pat. No. 3,345,673 describes an applicator especially adapted for use in applying cosmetics, medications, shoe polish or other fluids on the person, clothing, shoes, and the like of the user, and more particularly, to an applicator having a brush head adapted to be used in applying fluids in an efficacious manner.

### SUMMARY OF THE INVENTION

Many prior art methods to treat warts or other skin lesions are not effective or not effective enough. Further, some of the prior art methods need specific care and medical supervision, such as the treatment of warts with liquid nitrogen. Further, prior art devices showed to be instable.

Hence, it is an aspect of the invention to provide an alternative method and/or an alternative applicator, which preferably further at least partly obviate one or more of above-described drawbacks, and which are especially safe and durable.

In a first aspect, the invention provides an applicator for application of an acidic liquid, the applicator comprising in an embodiment a container containing said acidic liquid, the container comprising an opening in fluid contact with a nib for a controlled release of the acidic liquid, wherein the nib comprises a plurality of fibers and an adhesive material adhering the fibers. Herein, the nib is a part which may come into contact with an area (to be treated, such as tissue, mucus membrane, nail) in order to deliver a (safe and effective) dose of an active (medical) ingredient. Hence, the nib, and more in general the applicator or the liquid release unit is especially configured to deliver a (safe and effective) amount of a treatment liquid (also indicated herein as the acidic liquid).

In a specific embodiment, the invention provides an applicator, especially for application of (at least part of) an acidic liquid on a (part of a) body surface. Herein, the term “body surface” (herein also indicated as a topical area) refers to a surface of a body, such as the skin of a foot, an arm or a leg, but also, e.g., the lining or special skin (mucosa) inside a mouth and the mucus membranes and tissue at intimate areas. The term “body surface” or “topical area” may optionally also refer to a nail.

Hence, the invention provides an applicator for topical application of an acidic liquid (on topical areas). In an embodiment, the invention provides an applicator for topical application of an acidic liquid, the applicator comprising a container containing said acidic liquid, the container comprising an opening in fluid contact with (a liquid release unit comprising a) a nib for a controlled release of the acidic liquid, wherein the nib comprises a plurality of fibers and an adhesive material adhering the fibers.

With such applicator, in a safe and controlled way the acidic liquid can be applied to a topical area, by contacting the applicator, more precisely its nib, with the topical area.

The nib (also indicated herein as tip) of the device is configured to facilitate transport (through its capillaries) of liquid, especially acidic liquid, from (a surface of) the nib to a surface (of a topical area) in contact with (said surface of) the nib, wherein the amount of liquid released may be controlled by the contact time between the nib and the topical area and by the configuration of the nib.

The properties of the applicator, especially the properties of the nib may especially be selected to obtain a controllable release of the acidic liquid (when used for topical application of an acidic liquid). For instance, the size (area) of the nib arranged to contact the topical area (also indicated as “contact area of the nib”) may be configured to release the required amount of acidic liquid (in a given period of contact between the nib and the topical area). Next to that, the amount of fibers used, and the number and size of the capillaries between the fibers will affect the amount of released acidic liquid (in a given period of contact between the nib and the topical area). Also, material properties of the fiber and the acidic liquid (the interaction between the acidic liquid and the fibers) may affect the amount of released acidic liquid (in a given period of contact between the nib and the topical area). The transport and the release of the acidic liquid may especially be the result of the (physical) properties of the nib. Especially, no additional external forces, such as a pressure may need to be exerted (on the liquid and/or the container) to force a release of the liquid. The nib (comprising a liquid, especially in the capillaries) may only have to be positioned at a topical area to provide a release of the liquid. The nib may absorb the liquid. When not in use (used on a topical area) the liquid may thus not spontaneously be released.

By using the applicator as defined herein to apply the acidic liquid, the acidic liquid may safely and effective be applied targeted at substantially only the (topical) location to be treated, preventing damaging healthy locations that do not require any (peeling) treatment. The applicator, especially, may be arranged to deliver substantially the required dosage of the acidic liquid (on the topical location to be treated), avoiding an overdose (or insufficiency) of the acidic liquid that could lead to harming the skin instead of treating the skin. Hence, the present applicator, and also the herein proposed liquid release unit, allow a targeted application of the acidic liquid, thereby facilitation curing of e.g. a lesion, while substantially not affecting surrounding (healthy) tissue.

The invention also provides such acidic liquid per se, i.e. an acidic liquid especially for application of a topical treatment, such as a treatment of a skin disorder, a treatment of an oral health problem, a treatment of a topical problem at intimate areas, a skin removal (e.g. to remove a tattoo). The invention also provides such acidic liquid as defined herein, especially for use in acid treatment of a topological lesion, such as in a medical treatment. Further, the invention also provides such acidic liquid per se, especially for use in (acid) treatment of a topological lesion, such as in a cosmetic treatment. The invention also provides such acidic liquid, especially for use in (acid) treatment of a skin disorder, such as in a treatment of superficial lesions, in particular skin lesions, mucous membrane lesions and/or nail lesions, viral warts, verrucae, water warts (molluscum contagiosum), corns and calluses, skin hyperpigmentation (such as age spots, solar lentigo, senial lentigo), acne, keratosis pilaris, actinic keratosis, ingrown toenails, onychomycosis, eyelid xanthelasma, psoriasis, fungal nail infections, epidermodysplasia veruciformis, a HPV (human papillomavirus) caused skin disorder, scars, wrinkles, and melasma. The invention also provides such acidic acid liquid as defined herein, especially for use in (acid) treatment of topical oral health problems such as mouth ulcers (canker sores) and cold sores. The invention also provides such acidic acid liquid as defined herein, especially for use in (acid) treatment of topical problems at intimate areas, at mucus membranes and tissue, such as hemorrhoids, genital warts. The invention also provides such acidic liquid as defined herein, especially for use in (acid) treatment for skin removal purposes such as a tattoo and/or a skin tag.

The acidic liquid is especially applied to peel the skin or mucous membrane, especially the skin, by which the skin or mucous membrane may renew itself. The treatment liquid will have an advantageous effect on the lesion to be treated; the acidic liquid, especially the acid(s) in the liquid, may have a peeling effect, the liquid, especially the acid(s), may also provide an oxidation effect to the lesion.

The applicator may comprise different acidic liquids as one or more different liquids may be applied. Especially, the acidic liquid comprises one or more acids selected from trichloroacetic acid (TCA), formic acid (FA), and salicylic acid. The acidic liquid may (further) comprise one or more acids selected from acetic acid, hydrochloric acid (HCl), and nitric acid (HNO₃). Especially, the acidic liquid, such as comprised by the applicator, may comprise one or more acids selected from the group consisting of trichloroacetic acid, salicylic acid, formic acid, glycolic acid, dichloro-acetic acid, monochloroacetic acid, acetic acid, citric acid, ascorbic acid, boric acid, nitric acid, sulfuric acid, phosphoric acid, oxalic acid, lactic acid, hydrochloric acid.

The applicator or the acidic liquid, or applicator in combination with the acidic liquid, may be used for several types of lesions. The applicator or the acidic liquid, or applicator in combination with the acidic liquid, may be applied for a medical treatment and/or non-medical treatment, such as a cosmetic treatment. Herein, the term “acidic liquid” refers to the (treatment) liquid comprising the acid and optionally one or more other components (which may be applied on the topical area).

In a further aspect, the invention especially provides the use of the applicator, as defined herein, for a (cosmetic) treatment of a topical lesion selected from the group consisting of a superficial lesion, a skin lesion, a mucous membrane lesion, a nail lesion, a viral wart, verrucae, a water wart (molluscum contagiosum), a corn, a callus, an age spot, a solar lentigo, a senial lentigo, acne, keratosis pilaris, actinic keratosis, an ingrown toenails, onychomycosis, eyelid xanthelasma, psoriasis, a fungal nail infection, a skin tag, a mouth ulcer, a canker sore, a cold sore, a hemorrhoid, a genital wart, a tattoo, epidermodysplasia veruciformis, a HPV (human papillomavirus) caused skin disorder, a scar, a wrinkle, melisma, a tattoo, and a skin tag.

The term “wart” may e.g. relate to one or more of verruca vulgaris and verruca plantaris. The acidic liquid is especially suitable for the treatment of one or more of a wart and a corn. Likewise, also the acidic liquid per se may be used. Hence, the invention provides in yet a further aspect also the use of the acidic liquid as defined herein, for a (cosmetic) treatment (or a medical treatment) of a topical lesion selected from the group consisting of a superficial lesion, a skin lesion, a mucous membrane lesion, a nail lesion, a viral wart, verrucae, a water wart (molluscum contagiosum), a corn, a callus, an age spot, a solar lentigo, a senial lentigo, acne, keratosis pilaris, actinic keratosis, an ingrown toenails, onychomycosis, eyelid xanthelasma, psoriasis, a fungal nail infection, a skin tag, a mouth ulcer, a canker sore, a cold sore, a hemorrhoid, a genital wart, epidermodysplasia veruciformis, a HPV (human papillomavirus) caused skin disorder, a scar, a wrinkle, melisma, a tattoo, and a skin tag. In a further aspect, the invention provides an acidic liquid for treatment of a topical lesion selected from the group consisting of a superficial lesion, a skin lesion, a mucous membrane lesion, a nail lesion, a viral wart, verrucae, a water wart (molluscum contagiosum), a corn, a callus, an age spot, a solar lentigo, a senial lentigo, acne, keratosis pilaris, actinic keratosis, an ingrown toenails, onychomycosis, eyelid xanthelasma, psoriasis, a fungal nail infection, a skin tag, a mouth ulcer, a canker sore, a cold sore, a hemorrhoid, a genital wart, epidermodysplasia veruciformis, a HPV (human papillomavirus) caused skin disorder, a scar, a wrinkle, melisma, a tattoo, and a skin tag, wherein the acidic liquid is topically applied by contacting a topical area with the nib (of an applicator or liquid release unit), wherein the applicator (or liquid release unit) comprises said acidic liquid.

In an embodiment, the invention provides an acidic liquid for use in the treatment of a topical lesion selected from the group consisting of a superficial lesion, a skin lesion, a mucous membrane lesion, a nail lesion, a viral wart, verrucae, a water wart (molluscum contagiosum), a corn, a callus, an age spot, a solar lentigo, a senial lentigo, acne, keratosis pilaris, actinic keratosis, an ingrown toenails, onychomycosis, eyelid xanthelasma, psoriasis, a fungal nail infection, a skin tag, a mouth ulcer, a canker sore, a cold sore, a hemorrhoid, a genital wart, epidermodysplasia veruciformis, a HPV (human papillomavirus) caused skin disorder, scars, wrinkles, melisma, a tattoo, and a skin tag, wherein the acidic liquid is topically applied by contacting a topical area with the nib of a liquid release unit of a kit of parts comprising the liquid release unit and a container unit, and wherein the container unit comprises the acidic liquid.

In an embodiment, the acidic liquid especially is provided for use in the treatment of a wart or a corn. It may further be advantageous using the applicator according to the invention for applying acidic liquid given above.

The term topical treatment especially relates to the treatment with the acidic liquid to a body surface, such as the skin or a mucous membrane, especially the (human) skin.

In a specific embodiment, the applicator comprises the acidic liquid, wherein the acidic liquid has a pH less than 6, such as less than 4, like equal to or less than 1, even in some embodiments less than 0, such as in the range of −2-6, such as −1.5-5, especially such as −1.5-4. In yet a further embodiment, the acidic liquid may especially comprises an acid at a concentration in the range of 1-85 wt. %, such as 10-75 wt % (relative to the total weight of the liquid). The acidic liquid may also comprise two types of acids both at a concentration according to the above given ranges, such as a first acid at a concentration in the range of 10-40 wt % and a second acid at a concentration in the range of 50-85 wt. % (with the concentration of the multiple acids adding up to a concentration below 100 wt. %). The acids in the acidic liquid may especially have a pKa value selected from the range of −9.3−(+)14 (i.e. between 14 and −9.3), such as in the range of −6.3−(+)9.24 (i.e. between 9.24 and −6.3).

In yet another embodiment, the acidic liquid comprises at least one or more of trichloro acetic acid, salicylic acid, and formic acid. In a specific embodiment, the acidic liquid comprises at least one or more of trichloro acetic acid and formic acid.

Of course, the acidic liquid may optionally comprise one or more other ingredients, including one or more other active ingredients. The liquid (composition) as described herein may comprise other ingredients commonly used in cosmetics and pharmaceutical products, such as one or more of a surfactant, a colorant, a nail penetrating compound, a skin restoring compound, a microenvironment controlling compound, and a perfume. The liquid described herein is especially an aqueous liquid.

For instance, the acidic liquid may further include one or more excipients. An excipient is especially an inactive substance formulated alongside the active ingredient(s) of a product or medication, for the purpose of bulking-up formulations that contain such active ingredient(s). Excipients may for instance also be indicated as filler or diluent. Excipients may e.g. include one or more of binders, coatings, disintegrates, fillers, flavors, colorants, lubricants, glidants, sorbents, preservatives, sweeteners, etc. etc.

The acidic liquid may further e.g. comprise silk fibroin. Silk fibroin is a protein derived from hydrolization of silk fibers, naturally secreted by silk work Bombyx mori. Silk fibroin has diverse applications in the biomedical field, which can be attributed to its high tensile strength, controllable biodegradability, non-cytotoxicity, low-antigenicity and non inflammatory characteristics. The use of silk fibroin extract can aid the healing process during regeneration and repair of normal and functional nail tissue. The acidic liquid may further (also) comprise pentylene glycol. Pentylene glycol is used as moisturizing agent. It is a colorless liquid, very low in odor, that is both water and oil-soluble. Due to its unique molecular properties, including a well separated charge distribution pattern, pentylene glycol performs its moisturizing activity much better than comparable chemicals, i.e. propylene glycol. The acidic liquid may further (also) comprise dimethyl isosorbide. Dimethyl isosorbide is a delivery enhancer which can place active ingredients where they are needed most and it is thus used as penetrating system for the keratinous nail layer. Dimethyl isosorbide is a colorless liquid with excellent solvent properties. It enhances delivery of actives in the upper layers of the epidermis without promoting the product into the bloodstream. Moreover, dimethyl isosorbide improves stability of formulations, even those that are susceptible to hydrolysis and transesterification. The one or more of silk fibroin, pentylene glycol and dimethyl isosorbide may especially be applied in an acidic liquid for the treatment of nail fungus.

The acidic liquid may further e.g. comprise azone. Azone is a colorless to yellowish liquid comprising especially sodium hypochlorite as the active ingredient. Azone has been used to enhance percutaneous absorption. Its ability to improve penetration makes it an attractive compound for incorporation into the acidic liquid. Additionally or alternatively, pyrrolidones may be incorporated to enhance penetration. Also pyrrolidones show a high effect on hydrophilc diffusants and thus may function as a penetration enhancer in (human) skin.

The acidic liquid may further e.g. comprise a terpene. Terpenes are a large and diverse class of organic compounds, produced especially by a variety of plants. Many terpenes are hydrocarbons, but oxygen-containing compounds such as alcohols, aldehydes or ketones (terpenoids) are also found. Their building block is the hydrocarbon isoprene, CH₂═C(CH₃)—CH═CH₂. Terpene hydrocarbons therefore have molecular formulas (C₅H₈)ₙ, they are classified according to the number of isoprene units. Plant oils, which contain terpenes, have shown increasing promise in vivo, inhibiting multiple species of bacteria. For example, cinnamon oil has shown broad-spectrum activity against Pseudomonas aeruginosa. Also olive leaf oil has shown beneficial properties, especial with respect to the the micro-environment, having antimicrobial properties for some species as well as healing properties and supporting properties for other species. Cumene (isopropylbenzene) is a terpene that has been shown to comprise important anti-bacterial properties. Also limonene, carvone, and pinene have shown beneficial effects.

The acidic liquid may further e.g. comprise oxazolidones. The oxazolidinones are a class of antimicrobial agents which have a unique structure and good activity against gram-positive pathogenic bacteria. Oxazolidinones are a class of compounds containing 2-oxazolidine in the structure. Oxazolidinones represent a new class of synthetic antibacterial agents active against multiple-resistant gram-positive pathogens, including methicillin-resistant Staphylococcus aureus (MRSA), penicillin-resistant streptococci, and vancomycin-resistant enterococci. Hence, oxazolidones especially may be incorporated in the acidic liquid being able to control the microenvironment.

The acidic liquid may further e.g. comprise urea (or carbamide). Urea is an organic compound with the chemical formula CO(NH₂)₂. Urea-containing creams are widely known and used as topical dermatological products to promote rehydration of the skin. It further appears that urea can be indicated for psoriasis, xerosis, onychomycosis, ichthyosis, eczema, keratosis, keratoderma, corns and calluses. Its use in the e.g. the treatment and/or prevention of Athlete's foot may be intended as moisturizer agent for dry skin affected by athlete's foot. The acidic liquid may further e.g. comprise allantoin. Allantoin is a chemical compound with formula C₄H₆N₄O₃. It is also called 5-ureidohydantoin or glyoxyldiureide. It is a diureide of glyoxylic acid. It is used for its moisturizing properties since it increases the water content of the extracellular matrix. It also enhances the desquamation of upper layers of dead skin cells, favoring a faster healing process of damaged skin. The acidic liquid may further e.g. comprise panthenol. Panthenol is the alcohol analog of pantothenic acid (vitamin B5), and is thus a provitamin of B5. In organisms it is quickly oxidized to pantothenate. In e.g. the treatment (and/or prevention) of athletes's foot, panthenol may be used as a humectant, emollient and moisturizer. The one or more of urea, allantoin, and panthenol may especially be applied in the acidic liquid for the treatment (and/or prevention) of Athlete's foot, such as in the applicator.

Especially suitable additives included in the acidic acid may be selected from the group consisting of phenolic phytochemical compounds, fatty acids, anthraquinones, polysaccharides, oligomeric tannins, and monoterpene alcohols. Such compounds may especially be advantageous for care and skin lesions treatment. For instance, the acidic liquid may further include aloe vera. Yet in another embodiment, the acidic liquid may further include an extract selected from the group consisting of an algae extract, a cranberry extract, and an olive leaf extract.

Hence, the invention further provides an acidic liquid further comprising one or more of an azone, dimethylisosorbide, a oxazolidinone, a pyrrolidone, a terpenes, and urea. The acidic liquid especially comprises acidified water.

The applicator (and/or the liquid release unit) comprising the acidic liquid, especially the container and the (fibers of the) nib, especially comprises an acid resistant material. Especially good materials for the container and (the fibers of) the nib may independently be selected from the group consisting of acid resistant metals, chlorinated polyvinyl chloride (CPVC), ethylene chlorotrifluoroethylene (ECTFE), (enhanced) polytetrafluoroethylene, (enhanced PTFE), high-density poly ethylene (HDPE), polyether ether ketone (PEEK), polypropylene (PP), polysulfone (PSU), polyphenylene sulfide (PPS), polyvinyl chloride (especially type 1 (PVC, Type 1) or type 2 (PVC, Type 2)), polyvinylidene fluoride (PVDF), polytetrafluoroethylene (PTFE), Polyamide-imide (PAI), ultra high molecular weight polyethylene (UHMW) and also coated (with an acid resistant material, especially an acid resistant polymer) metals like aluminum. Yet in a further embodiment, good materials for the container and/or the fibers of the nib (and/or the liquid release unit) may independently be selected from the group consisting of high-density poly ethylene (HDPE), low-density poly ethylene (LDPE), polyamide (PA), polycarbonate (PC), polyethylene terephthalate (PET) (including e.g. polyethylene terephthalate glycol PETG), polymethylpentene (PMP), polyoxymethylene (POM), polypropylene (PP), polystyrene (PS), polysulfone (PSU), polyvinyl chloride (especially rigid (PVC HART) and flexible (PVC WEICH), styrene acrylonitrile (SAN), ethylene chlorotrifluoroethylene (ECTFE), ethylene chlorotrifluoroethylene (ETFE), fluorinated ethylene propylene (FEP), polytetra-fluoroethylene (PTFE), polyvinylidene fluoride (PVDF), ethylene propylene diene rubber (EPDM), fluorocarbon rubber (FPM), and acrylonitrile (nitrile) butadiene rubber (NBR). These materials are especially resistant against the acidity of the acidic liquid. It has been found that especially polypropylene (PP) and polyethylene terephthalate (PET) (such as e.g. polyethylene terephthalate glycol (PETG)), do have good acid resistence against the acids comprised in the acidic liquid according to the present invention where as they also show different resistency for different acids, see for instance the table below showing the chemical resistance and processability in a nib comprising fibres of some polymeric materials.

| Polymeric | resistance against | resistance against | processability in |
|---|---|---|---|
| material | formic acid | (trichloro-) acetic acid | (fibers of) a nib |
| PP | +/− | ++ | ++ |
| PET | ++ | +/− | ++ |
| PE | ++ | +/− | + |
| PC | +/− | ++ | +/− |
| PTFE | ++ | ++ | + |
| PA | +/− | − | + |

For instance, formic acid in the acidic liquid may negatively affect polypropylene, while polyethylene terephthalate is more resistant to formic acid. In contrast to that, polypropylene may chemically be resistant towards trichloroacetic acid or acetic acid. Hence, when the acidic liquid only comprises trichloroacetic acid, the fibers and/or the container may be made of polypropylene, whereas it may be advantageous to use fibers and/or a container made from polyethylene terephthalate when the acidic liquid is substantially formic acid. Hence in embodiments, the applicator comprises a polymeric container, wherein the polymeric container comprises a container wall comprising polypropylene (PP), and wherein the fibres comprise polypropylene (PP) comprising fibres. Especially such embodiment may contain an acidic liquid comprising trichloroacetic acid.

In further embodiments, the applicator comprises a polymeric container, wherein the polymeric container comprises a container wall comprising polyethylene terephthalate (PET), and wherein the fibres comprise polyethylene terephthalate (PET) comprising fibres. Especially such embodiment may contain an acidic liquid comprising (being) substantially formic acid.

Hence in embodiments, the applicator comprises a polymeric container, wherein the polymeric container comprises a container wall comprising a polymer material selected from the group containing polypropylene (PP) and polyethylene terephthalate (PET), and wherein the fibres comprise fibres selected from the group of fibres comprising polypropylene (PP) comprising fibres and polyethylene terephthalate (PET) comprising fibres.

Different types of nibs may be applied varying in their purpose, shape and size, as well as the material they are made from to be resistant to the exposure acidic compounds. The nib of the applicator comprises a plurality of fibers, wherein the fibers are adhered together by an adhesive material. Especially, the adhesive material is applied to adhere the fibers while keeping open channels between the fibers to enable transport of the acidic liquid between the fibers. Hence during the production process of the nib, especially the adhering of the fibers part of the process, an adhesive material should be used that does not block all channels between the fibers.

In an embodiment, the nib comprises a bundle of parallel fibers held together by partial adhesion. In an embodiment, the nib comprises an elongate bundle of fibers extending parallel to the axis of elongation. Especially, the fibers define capillary passageways there between through which the acidic liquid may be transported. Due to its relatively simple construction, the nib of the present invention can be produced continuously and in large quantities mechanically and therefore can be provided cheaply.

Amongst others, the nib of the present invention may be prepared by arranging fibers in parallel, securing the fibers in that arrangement by means of an adhesive material to form a coherent elongated bundle in which capillary passages between the fibers are provided (providing an—overall—porosity of the nib). The formed bundle is then cut to length and optionally narrowed at one (or both) of the longitudinal ends using, for example, a grinder. Further, e.g. one side may be processed to provide a slanted face (herein also indicated as smoothened surface or oblique surface). Hence, in an embodiment a fibrous axially elongated nib is provided, with the fibers of the nib extending axially in a bundle. The fibers being secured to each other so that the bundle is coherent and the fibers define capillary passageways between them. Hence, especially the nib comprises a bundle of fibers with adhesive material circumferentially surrounding the bundle over at least part of a bundle length. Alternatively or addition to the circumferentially surrounding adhesive material, the fibres may also be glued together, e.g. with the herein described resins. Yet alternatively or additionally, the fibers may (partly) melted together. Hence, the nib may included fibers which are over at least part of their length melted together. Instead of the terms “melted” or “melted together”, also the term “fused” may be used. Such melt connections or welding connections may e.g. be obtained by using a solvent, such as cyclohexanone. Hence, the nib may include melt connections between fibers. Even more especially, the nib may thus include solvent welding connections between fibers. Such solvent welding connections are especially only over part of the length of the fibers. Further, the solvent welding connections may include the same polymeric material as of the fibers, and may further optionally include a solvent, such as cyclohexanone (which may be incorporated in the welding connections).

Where the fiber bundle is made coherent by an adhesive material, it is advantageous that the adhesive material be used in an amount as small as possible while being sufficient to maintain the configuration of the fiber bundle in a coherent, elongated rod shape. Furthermore, the nib of the invention may have a transverse cross section which is substantially circular, oval, triangular, tetragonal (square), polygonal or star shape, especially however circular or tetragonal, even more especially circular.

In addition and especially, also the adhesive material should be resistant to the acidic fluid. Especially good materials for the adhesive material may be selected from the group of expo resins and acrylic resins. Examples of expoxy resins are bisphenol A epoxy resins (such as expoxy bisphenol A vinyl ester, and urethane epoxy bisphenol A vinyl esters), bisphenol F epoxy resin, glycidylamine epoxy resins, novolac epoxy resins, and aliphatic epoxy resins. Examples of acrylic resins are is polymethyl methacrylate resins (PMMA), ethyl methacrylate resins, butyl methacrylate resins. A plurality of resins were tested, but these resins listed here especially appeared to be able to provide a good and stable nib, being stable against the acids used, and providing a reliable flow through the nib.

Especially an adhesive material comprising a phenoxy resin may resist the acidic liquid comprising a pH<0 and may effectively adhere the fibers without blocking all channels between the fibers. Examples of phenoxy resins (polyhydroxyethers) are unmodified bisphenol-A type phenoxy resins, bisphenol-A/bisphenol-F type phenoxy resins, phenoxy-polyester hybrids, and caprolactone-modified phenoxy resins. Hence in an embodiment, the adhesive material comprised by the applicator comprises one or more of an epoxy resin and an acryl resin. In yet a further embodiment, the adhesive material comprises a phenoxy resin.

Suitable resins may e.g. be selected from Atlac 5200F, Atlac 4010, Atlac 382, Atlac 580, Atlac 430, Atlac E-Nova FW 1045, Atlac 590, from DSM (DSM composite resins AG). Other suitable resins may e.g. be selected from F07, F010, F013, F085, F086, F282, K022, K026, K095, and K190, from AOC (Vipel).

Trichloroacetic acid (TCA) in the acidic liquid as described herein, proved to be effective against a plethora of skin lesions, in particular warts, corn and calluses, molluscum contagiosum, acne, skin hyperpigmentation, actinic keratosis as well as nail lesions including ingrown toenails and onchyomycosis, and lesions in mucous membranes such as mouth ulcers and cold sores. Application of trichloroacetic acid is particularly effective against genital warts, and especially against ano-genital warts where it has been studied in comparative trials where its effectiveness was compared with other anti-wart therapies. In addition, TCA application was effective towards various other skin disorders, in particular: epidermodysplasia veruciformis-skin disorder also caused by HPV virus, acne, to remove acne scars and wrinkles, tattoo removal, eyelid xanthelasma, age spots, senial lentigo and solar lentigo, melasma. Additionally or alternatively, the acidic liquid may comprise one or more of formic acid and salicylic acid, which alone or in combination may also provide a good result. Hence, in an embodiment the acidic liquid comprises TCA, and optionally formic acid, in another embodiment the acidic liquid comprises formic acid, and optionally TCA, in yet another embodiment the acidic liquid comprises formic acid, TCA and salicylic acid.

Salicylic acid has a positive effect in treating skin and nails. In addition to that, salicylic acid gives a mild anesthetic effect. Salicylic acid in combination with TCA showed a synergistic effect against skin lesions and nail lesions. Another advantage is that using a combination of salicylic acid and TCA allows for a composition with a relatively low concentration of each of salicylic acid and TCA with a similar effect to compositions using only TCA or only salicylic acid. Using the relatively low concentrations of TCA and salicylic acid decreases the chance of skin irritation due to either of these compounds. Moreover, salicylic acid diminished the discomforting burning feeling on skin and nails sometimes experienced by persons treated with products containing substantial amounts of TCA.

The acidic liquid and the applicator comprising the acidic liquid may thus amongst others be used in for instance a skin peeling treatment, for the medical or cosmetic treatment of skin lesion selected from the group consisting of warts, corn and calluses, and for nail treatments including ingrown toenails. A postulated mechanism of action is that the composition comprising TCA, or another acid, softens the skin or nail, and enables to peel the skin or nail lesion away. For severe lesions, multiple treatments may be needed. Both cosmetic and medical treatments may be performed with the acidic liquids according to the invention. Other active ingredients contributing to the treatment of the lesion may be added. For instance salicylic acid (see also above) is another component effective in corroding the skin or nails, and may be used as an additional active ingredient in combination with trichloroacetic acid. Especially, the applicator, liquid release unit, and the acidic liquid are used for a skin peeling treatment or the treatment of a skin lesion selected from the group consisting of warts, corn and calluses. The acidic liquids are particularly effective against warts and related lesions, including viral warts, verrucae, and water warts (molluscum contagiosum). These treatments are typically considered cosmetic treatments rather than medical treatments. The invention also provides the use of an acidic liquid as described herein for the cosmetic treatment of nails. In particular nail deformations (for instance as a result from onchyomycosis) and ingrown toenails may be treated effectively.

The invention further provides the use of an acidic liquid or treatment liquid as described herein for the treatment of a topical lesion. The invention further provides the use of the acidic liquids or treatment liquid as described herein for the cosmetic treatment of superficial lesions, such as selected from the group consisting of viral warts, verrucae, water warts (molluscum contagiosum), corns and calluses, and age spots, solar lentigo, senial lentigo, acne, mouth ulcers (canker sores), cold sores ingrown toenails, etc.

The acidic liquid especially comprises an aqueous liquid, such as especially water, and the acid. Especially, the total amount of other components in the acidic liquid, other than the acid and the liquid is less than 10 wt. %, especially less than 5 wt. %, examples of other compounds, such as a colorant, surfactant, an ingredient affecting the microenvironment or a perfume, are mentioned above. Especially, the treatment liquid comprises one or more of the acids in a concentration up to 85 wt. %, such as at maximum 75 wt. %, even for some acids more especially at maximum 45 wt. %. Higher acid concentrations may lead to undesired topological effects and/or may be unsafe, such as in the kind of application with children or specific topical parts, like mucous membrane. In a specific embodiment, the treatment liquid comprises the acid in a concentration in the range of 1-85 wt. %. Lower concentrations than 0.5 wt. % may not be effective enough. Especially, the concentration of the acid in the treatment liquid is at least 1 wt. %.

The applicator allows the treatment of the topological lesion by local contact of the applicator on the skin. This is safer and also more effective than applicators that need contact with al larger region than lesion or applicators that serve more than the lesion. The applicator may especially be configured to provide the acidic liquid to a local topical area of ranging from 0.01-4 cm², such as 0.02-2.5 cm², such as 0.1-1.0 cm², like 0.1-0.5 cm², by means of a direct contact. Hence, in an embodiment, the nib (of the applicator) comprises a (smoothened) surface having a surface area (also indicated as contact area of the nib) in the range of 0.01-4 cm², like e.g. 0.02-2.5 cm².

In a further embodiment, the applicator comprises a grip part. Hence, in an embodiment, the applicator comprises a nib comprising a smoothened surface having e.g. a surface area in the range of 0.01-4 cm², such as 0.02-2.5 cm², and wherein the applicator comprises a grip part, wherein the physical topical application includes touching a topical area and keeping the nib in physical contact with the topical area in the range of 0.2-5 seconds.

In an embodiment, the applicator comprises a nib comprising a smoothened surface having a surface area in the range of 0.01-0.1 cm². In yet another embodiment the applicator comprises a nib comprising a smoothened surface having a surface area in the range of 1-2.5 cm². Especially for acidic liquid comprising a very low pH a small surface area may be advantageous, whereas for acidic liquids comprising a higher pH (although still acidic) a larger surface area (and a longer contact time between the topical area and the nib surface) may be advantageous. In embodiments, a specific dimension of the nib, especially the width or the diameter of the nib, is selected in the range of 0.5 mm-15 mm, such as 2 mm-10 mm, especially 3 mm-10 mm, such as 3 mm-6 mm, and even more especially 4-5 mm. The nib may e.g. have a diameter in the range of 0.5-10 mm, such as 1-6 mm.

In an embodiment of the treatment, the physical topical application comprises one or two sequential touchings of the topical area. In another embodiment of the treatment, the physical topical application comprises one touching of the topical area during 4 sequential days followed by a period of 7 days no touching of the topical area (with the nib) and optionally repeating the sequence of 4 days touching and 7 days no touching of the topical area with the nib. All kind of schemes, amongst others dependent upon e.g. the to be treated lesion, the age of the person, etc., may be chosen.

As will be known by the person skilled in the art, the total amount of acidic liquid supplied to a topical region can be controlled by matching the contact area between the nib and the topical area, which is controlled by the cross sectional area of the nib (and thus by the total number and diameter of the fibers) as well as by the final cut of the nib, as round nibs may be cut very slanting ending up with a relative large contact area, and by the dosage, i.e., the flow of acidic liquid per square mm of contact area as well as the total contact time (between the nib and the topical area). It was found that for the average size of lesions require an amount of acidic liquid in the range of 25 nl to 0.25 μl. Most conveniently, the application device is placed on the topical area are for about 1 to 10 seconds. Hence in an embodiment the nib is configured for flow of the acidic liquid, when the pen is configured vertical with the nib down, in the range of 0.1-10 ml/h. Herein, terms like “touching” and “placing on an area” especially indicates the contact of the applicator or liquid release unit without human force. Hence, the applicator or liquid release unit may rest on the topical area, and the pressure experienced by the topical area is substantially only the weight of the applicator or liquid release unit.

In view of safety, especially child safety, the applicator may in an embodiment include an infant opening protection. For instance, the infant opening protection system may include a turning system that has a state wherein a cap on the applicator is blocked and a state the cap may be removed from the applicator to disclose (and use) the nib. In this respect, in embodiments, the applicator in configured in such a way that the nib may not be removed or separated from device, especially the container and no acidic liquid may leak or flow from the applicator in an uncontrolled way.

In a further embodiment the applicator device comprises a pen (or is pen shaped) and the liquid release unit comprises a nib, wherein both devices are integrated. In yet another embodiment the container unit comprises a tube, and the liquid release unit comprises a nib for a controlled release of the acidic liquid, wherein the nib of the liquid release unit is in contact or integrated with the tube. Hence, in these embodiments the applicator is an integrated device (optionally with e.g. a closure, etc.). However, the applicator may also consist of two or more parts, that can be dissociated from each other in a functional way. For instance, a liquid release unit, such as a kind of cotton bud (comprising the nib), may be used to humidify with the acidic liquid from a container unit (comprising said acidic liquid).

The container unit may include a container containing said acidic liquid. However, the container unit may also include an absorbing material, completely soaked with the acidic liquid. Such absorbing material will especially be in physical contact with the nib, for transport of the acidic liquid to the nib.

The nib is especially designed as wick. Especially (capillaries in) the nib may allow a liquid to flow (spontaneously) through the nib when the nib provides a fluid connection between a liquid reservoir (such as a container) and a contact area, such as a topical area.

Assuming the applicator having a pen shape, the diameter over the length of the pen may be substantially round. However, especially the applicator comprises a grip part. Such grip part may e.g. comprise a triangular cross-section, especially having rounded angles. Hence, in yet an even more specific embodiment, the applicator comprises different cross-sections, gradually changing from round to a grip part having a triangular cross-section (and optionally turning to a substantially round cross-section again). The applicator may include a closure. When defining the shape of the applicator, such closure may be included.

Hence, it is a further aspect of the invention to provide a kit of parts comprising a liquid release unit and a container unit for topical application of an acidic liquid, wherein the container unit comprising a container containing said acidic liquid and the liquid release unit comprises a nib for a controlled release of the acidic liquid and wherein the nib comprises a plurality of fibers and an adhesive material adhering the fibers (and wherein during or before normal operation the container unit is in contact with the the liquid release unit). The kit of parts may especially be used to assemble the applicator as described herein.

In an embodiment, both the liquid release unit and the container unit are integrated in the applicator according to the present invention, but the container unit and liquid release unit can be separated from each other. In yet another embodiment the container unit comprises a (glass) bottle and the liquid release unit comprising the nib may e.g. be dipped in the container unit (to provide such nib comprising the acidic liquid for release to a topical area). In yet another embodiment the liquid release unit is in fluid connection with a fluid transporting means selected from the group of hollow transporting means such as a straw, a tube, a capilary, etc., and the container unit is selected from the group of container units comprising among others a glass bottle, a polymer bottle, a cup, etc. In yet another embodiment the nib is connected to a cotton but and may e.g. be dipped in a container unit comprising a container, such as a cup, a bottle, a glass, a tube, etc. In a further embodiment, the acidic liquid is comprised in an aerosol, wherein the aerosol may be contacted with the liquid release unit (and subsequently, the nib comprised by the liquid release unit is contacted with the topical area to be treated). In yet another embodiment, the liquid release unit comprises a patch, wherein the nib comprised by the liquid release unit is in previously provided with the acidic liquid by the container unit. The patch may (slowly and) in a controlled way releases the acidic liquid to the topical area.

Hence, in an embodiment the liquid release unit may comprises a tissue contact element selected from the group consisting of an adhesive superstrate, a wound bandage, a wound coating, and a patch, wherein the tissue contact element comprises said nib. The liquid release unit, or more especially the nib, may be provided with the acidic liquid by contacting the nib with the acidic liquid, such as by spraying, etc. In embodiments, the nib may be attached on to a pen, a tube, a pumper, an aerosol sprayer, a stick, a patch, a cotton bud, a straw (into a glass bottle), etc. Especially the nib is the part which comes into contact with tissue, mucus membrane, nail or other to be treated area, in order to deliver a safe and effective dose of an active medical ingredient. Different types of nibs vary in their purpose, shape and size, as well as the material they are made from to be resistant to the exposure acidic compounds.

In yet a further aspect, the invention also provides a method of providing the (applicator) nib as defined herein. The nib as defined herein especially comprises fibers that are adhered together by an adhesive material. The fibers may be configured in a pultrusion process from polymeric (such as polypropylene, polyethylene terephthalate, etc., see above) yarn being woven into a larger string, or bundle of fibers. Pultrusion is a manufacturing process in which normally reinforcing fibers impregnation with matrix is pulled through a die to form composites of a constant cross-section. In the method described herein, the fibers in the bundle, especially the outer surface (but not the ends) of the bundle of fibers, is contacted with a resin (such as an epoxy resin, an acrylic resin, or a phenoxy resin) to adhere the fibers. Contacting may especially be performed by spraying the bundle with the liquid resin or e.g. by pulling the bundle through a bath filled with the liquid resin. Successively, the fibers in the bundle are forced towards each other by pulling the bundle through a (round) opening (a die) and successively curing the resin resulting in a fixated bundle that finally may be cut in small pieces or nibs. Optionally the nibs may be grinded to obtain the desired contact surface. Especially by controlling the number of fibers, the diameter of the fibers, the length of the fibers, and the size (diameter) of the opening, the void fraction or capillary space in the bundle and thus the final nib may be controlled. Since transport speed through a nib will be affected by the capillary space (and hence the—overall-porosity), the length of the capillaries, as well as the fluid properties (such as surface tension and viscosity), the method of present invention may enable us to match the nib properties with the acidic fluid that will be used in the topical application. In embodiments, the length of the nib is selected in the range of 5-100 mm, such as 10-75 mm. Especially the length of the nib may be selected in the range of 20-50 mm, especially a nib comprising a width or diameter in the range of 4-5 mm. In embodiments, the porosity of the nib is selected to be >20% (volume %). In further embodiments, the porosity of the nib is selected to be larger than 40%, such as at least 50%. In even further embodiments, the porosity of the nib is selected to be 80% at maximum, such as 75%. Especially, the porosity of the nib is selected in the range of 60-70 vol. %.

Hence, the invention further provides a method for providing a nib applying a pultrusion equipment, wherein the pultrusion equipment comprises a pulling device and an opening; the method comprising: (i) providing a polymeric yarn and arranging the yarn into a bundle of yarn comprising an outer surface and a diameter of the bundle of yarn; (ii) providing a resin to the outer surface of the bundle of yarn and passing this bundle of yarn through the opening, wherein the diameter of the opening is smaller than the diameter of the bundle of yarn, to compress the bundle of yarn; (iii) curing the resin in the bundle of yarn providing a fixated bundle of fibers; and (iv) providing a nib by cutting a part of the fixated bundle of fibers in a direction perpendicular to the outer surface of the fixated bundle of fibers, and optionally grinding the nib.

The term “substantially” herein, such as in “substantially consists”, will be understood by the person skilled in the art. The term “substantially” may also include embodiments with “entirely”, “completely”, “all”, etc. Hence, in embodiments the adjective substantially may also be removed. Where applicable, the term “substantially” may also relate to 90% or higher, such as 95% or higher, especially 99% or higher, even more especially 99.5% or higher, including 100%. The term “comprise” includes also embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices herein are amongst others described during operation. As will be clear to the person skilled in the art, the invention is not limited to methods of operation or devices in operation.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

The invention further applies to a device comprising one or more of the characterizing features described in the description and/or shown in the attached drawings. The invention further pertains to a method or process comprising one or more of the characterising features described in the description and/or shown in the attached drawings.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1a and 1b schematically depict an embodiment and some aspects of the applicator;

FIG. 2 schematically depicts an embodiment of the kit of parts;

FIG. 3 schematically depicts an embodiment of a patch as liquid release unit; and

FIG. 4 schematically depicts an embodiment of a pen applicator.

### DETAILED DESCRIPTION OF THE EMBODIMENTS

The invention especially provides a nib as part of a medical device (an applicator for applying the treatment liquid to the treatment tissue/area) for the treatment of superficial lesions, in particular skin lesions, mucous membrane lesions and/or nail lesions, an applicator comprising a treatment liquid/active ingredient(s) and the configuration of the nib enables the use of the treatment liquid/active ingredient(s) onto tissue in a controlled way. The configuration of the nib enables a capillary effect, by comprising air channels in longitudinal direction in which a plurality of treatment liquid/active ingredient(s) passages. The configuration of the nib is chemically resistant to the active ingredients used in the treatment liquid/active ingredient(s); the materials used are selected based on the treatment liquid, especially regarding the acidity of the acidic treatment liquid. The treatment liquid(s) comprises an effective amount of active ingredient/acid, and an acceptable solvent, and is effective against a plethora of superficial lesions selected from the group consisting of viral warts, verrucae, water warts (molluscum contagiosum), corns and calluses, skin hyperpigmentation (age spots, solar lentigo, senile lentigo), acne, keratosis pilaris, actinic keratosis, mouth ulcers (canker sores), cold sores, ingrown toenails, onychomycosis, eyelid xanthelasma.

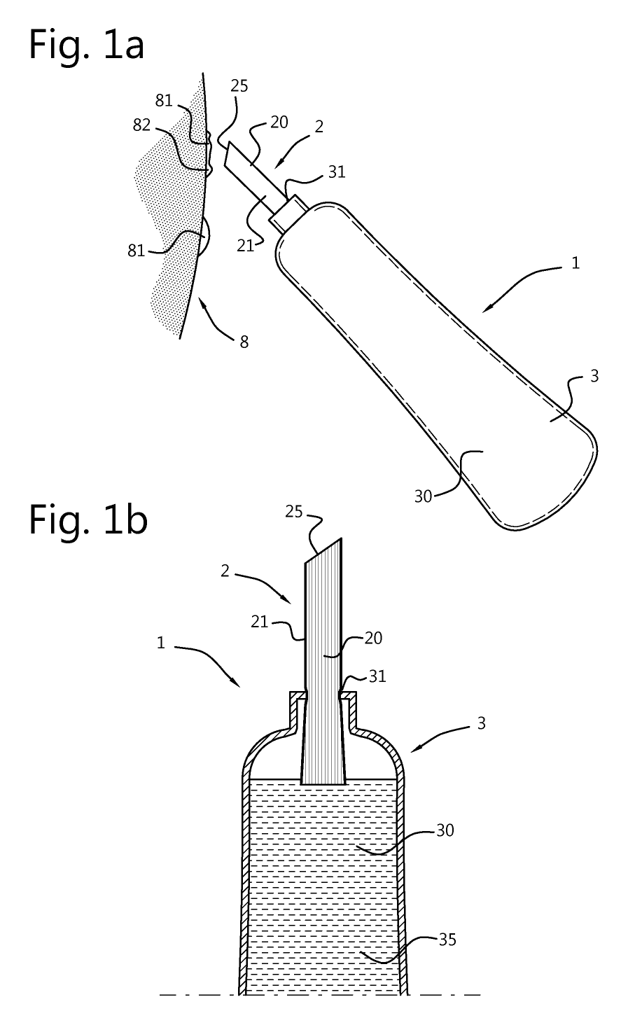

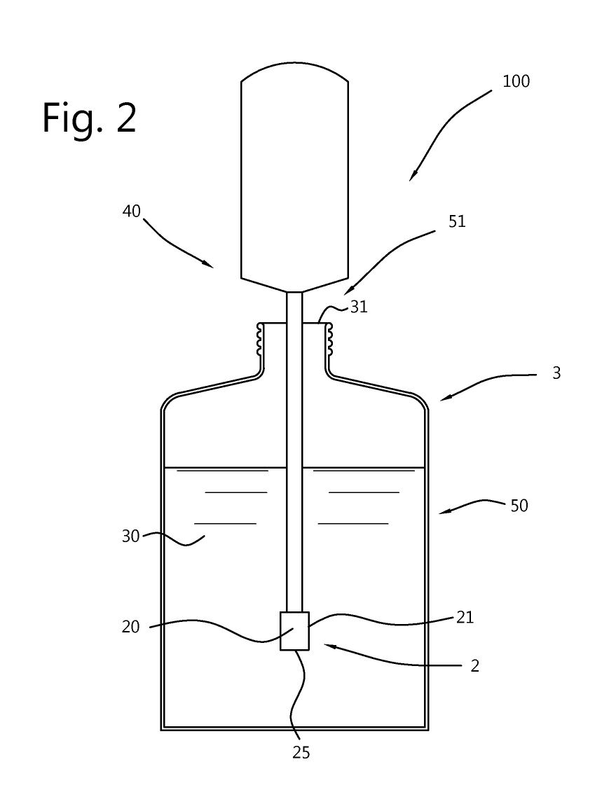

FIG. 1a schematically depicts an embodiment of an applicator 1. This embodiment of the applicator is especially a pen applicator, wherein a nib 2 is in arranged extending from the container 3. This is just one possible embodiment; in other embodiments the nib may for instance also be curved or the container may be configured in a complete different way. Also embodiments having a flexible connection between the container 3 and the nib 2 are anticipated. In the (pen) applicator 1, the nib 2 is in fluid connection with the acidic liquid 30 comprised (or contained) in the container. For this, the nib 2 is arranged through opening 31 of the container 3. The nib is made up from many fibers 20 adhered together by an adhesive material 21, such as a resin. In the production process of the nib 2 especially a bundle of fibers 20 is contacted with the adhesive material in such a way that the adhesive material substantially only covers the outer surface (see also FIG. 1b, showing a cross section of the embodiment of FIG. 1a) of the bundle of fibers, and the adhesive material substantially does not fill the voids or capillaries between the fibers. Based on these capillaries the acidic liquid 30 in the container 3 may be transported to the tip of the nib 2, especially the surface 25 of the tip of the nib. By contacting a body part 8, especially a lesion 81, such as a wart 82, an age spot, or a fungal nail, etc., a controlled (and targeted) release of acidic fluid 30 is provided, and the topical lesion may be treated (and eventually cured).

FIG. 1b schematically depicts a cross section of (part of) the applicator 1 also depicted in FIG. 1a. Schematically the fibers 20 are depicted (actually only a very small portion of all fibers are depicted for clarity reasons) in the nib 2. The fibers are kept together by the adhesive material 21, basically providing the outer surface of the nib 2. The acidic liquid 30 is contained in the container 3, wherein optionally a matrix 35, such as a sponge type matrix, is placed to hold the liquid. The nib 2 is in fluid contact with the acidic liquid 30, wherein the nib 2 is arranged in the container via an opening 31. Especially the acidic liquid is continuously in contact with the container and with the fibers (and the adhesive material). Therefore, especially the materials have to be selected to be compatible with the acidic liquid. For instance, formic acid in the acidic liquid may negatively affect polypropylene, while polyethylene terephthalate is more resistant to formic acid. In contrast to that, polypropylene may chemically be resistant towards trichloroacetic acid. Hence, if the acidic liquid only comprises trichloroacetic acid the fibers the container may be made of polypropylene, whereas it may be advantageous to use fibers and a container made from polyethylene terephthalate if the acidic liquid is substantially formic acid. Of course, also the adhesive material has to be selected depending on the properties (composition) of the acidic liquid to prevent the nib to fall apart (because of a (slow) reaction between the acidic liquid and the adhesive material).

FIG. 2 schematically depicts an embodiment of the kit of parts 100 for topical application of the acidic liquid 30. The kit comprises a liquid release unit 40 and a container unit 50. The acidic liquid 30 is contained in the container 3. The liquid release unit 40 comprises the nib 2. In the figure, the nib 2 may be dipped in the acidic liquid 30, such as to completely fill (saturate) the nib, especially the cavities between the fibers 20 of the nib. After filling the nib 2, the liquid release unit 40, especially the nib 2 can be used for topical application of the acidic liquid, e.g. by placing the tip surface 25 on a wart or a callus, etc. Surface 25 may be configured to the topical region to be treated. For instance the oblique tip surface 25 in the embodiment given in FIG. 1a-1b may be easier to use on toenails, whereas the flat surface 25 in FIG. 2 may be more appropriate to treat callus. It should be remarked that in the given embodiment, the liquid release element 51 comprised by container unit 50, especially comprises the container opening 31. In another embodiment, a specific liquid release element may be present. For instance, in an embodiment where the container unit comprises an aerosol pump and the acidic liquid is sprayed at the nib, the liquid release element may essentially comprise a nozzle.

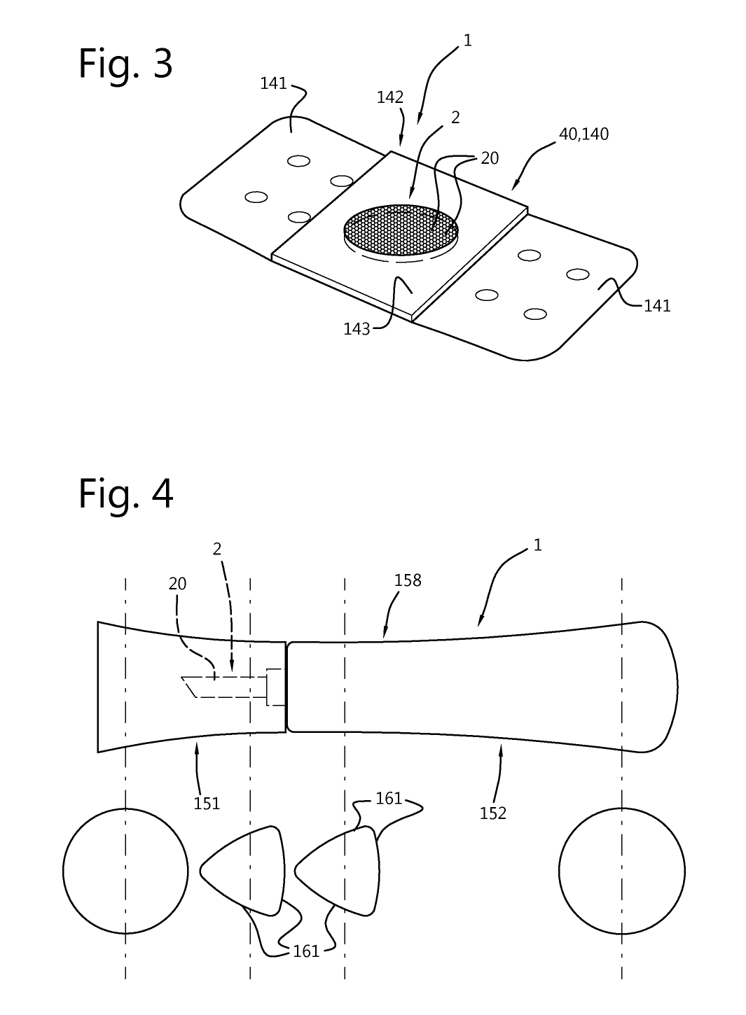

FIG. 3 schematically depicts an applicator 1, especially a liquid release unit 40 for topical application of the acidic liquid. The embodiment of the liquid release unit 40 is configured as a patch 140 that can be easily positioned at a body part, especially at a topical area to be treated, by means of two adhesive parts 141. The patch 140 further comprises a bandage part 142, in the center of the bandage part provided with the nib 2. The (fibers 20 of the) nib 2 may have been contacted with an acidic liquid before contacting a topical area, and thus may contain the acidic liquid. The liquid release unit 40, especially the nib 2, may release the acidic liquid at a topical area to be treated at the moment the patch 140 is applied to the area. The patch type embodiment especially is configured to be applied for a longer period. Therefore, especially the acidic liquid may be diluted (more) resulting in a higher pH of the acidic liquid, and the contact surface between the nib and the topical area may be selected to be relatively large (e.g. with respect to an embodiment of a pen type applicator as schematically shown in FIG. 4).

FIG. 4 schematically depicts an embodiment of the applicator 1, configured like a pen type embodiment. The pen 1 comprising a cover part 151 especially arranged to cover the nib 2 when the applicator is not in use, for instance to prevent the acidic liquid from (slowly) evaporating. However, in the schematically depicted embodiment the cover part 151 may be configured to prevent a child from removing the cover part from applicator 1. This may be especially advantageous to prevent children from getting in contact the acidic liquid. The second part 152 (or main part, to which the nib in this embodiment is associated) comprises a grip part 158. The grip part 158 especially has been configured to comfort handling the applicator 1 during topical application of the acidic liquid. The grip part 158 in combination with the facers 161 configured at the covering part 151 also support removing the cover part 151 from the second part 152, especially if the applicator 1 is designed to obstruct (in case of an infant opening protection) removing the cover part 151 from second part 152 of the applicator 1. Here, the grip part 158 shows another cross-section than the remaining part(s) of the applicator 1. For instance, the grip part 158 comprises a triangular type cross-section, whereas the remainder comprises a circular type cross-section. Especially, there is a gradual change between the types of cross-sections. The cross-sections are schematically depicted below the pen applicator 1.

1. An applicator for topical application of an acidic liquid, the applicator comprising a container containing said acidic liquid, the container comprising an opening in fluid contact with a nib for a controlled release of the acidic liquid, wherein the nib comprises a plurality of fibers and an adhesive material adhering the fibers, wherein the nib comprises an elongated bundle of said fibres extending parallel to an axis of elongation, wherein the acidic liquid has a pH less than 6, wherein the acidic liquid comprises an aqueous liquid, wherein the acidic liquid comprises at least one or more of trichloro acetic acid and formic acid, wherein the applicator comprises a polymeric container, and
(i) wherein the polymeric container comprises a container wall comprising polypropylene, and wherein the fibres comprise polypropylene comprising fibres; or
(ii) wherein the polymeric container comprises a container wall comprising polyethylene terephthalate, and wherein the fibres comprise polyethylene terephthalate (PET) comprising fibres; or
(iii) wherein the polymeric container comprises a container wall comprising polytetrafluoroethylene, and wherein the fibers comprise polytetrafluoroethylene comprising fibers.

2. The applicator according to claim 1, wherein the polymeric container comprises a container wall comprising polypropylene, wherein the fibres comprise fibres comprising polypropylene, and wherein the acidic liquid comprises trichloro acetic acid.

3. The applicator according to claim 1, wherein the polymeric container comprises a container wall comprising polyethylene terephthalate, and wherein the fibres comprise polyethylene terephthalate comprising fibres, and wherein the acidic liquid comprises formic acid.

4. The applicator according to claim 1, wherein the acidic liquid further comprises one or more acids selected from the group consisting of salicylic acid, glycolic acid, acetic acid, monochloro acetic acid, dichloro acetic acid, citric acid, ascorbic acid, boric acid, nitric acid, sulfuric acid, phosphoric acid, oxalic acid, lactic acid, and hydrochloric acid.

5. The applicator according to claim 1, wherein the acidic liquid has a pH of 4 or smaller, and wherein the acidic liquid comprises an acid at a concentration in the range of 1-85 wt. %.

6. The applicator according to claim 1, wherein the acidic liquid further comprises water and one or more of an azone, a dimethylisosorbide, an oxazolidinone, a pyrrolidone, a terpene, and urea.

7. The applicator according to claim 1, wherein the adhesive material comprises one or more of an epoxy resin and an acrylic resin.

8. The applicator according to claim 1, wherein the adhesive material comprises a phenoxy resin.

9. The applicator according to claim 1, wherein the nib is configured for flow of the acidic liquid, when the applicator is configured vertical with the nib down, in the range of 0.1-10 ml/h.

10. The applicator according to claim 1, wherein the applicator is pen shaped, wherein the nib comprises a smoothened surface having a surface area in the range of 0.01-2.5 cm², wherein the applicator comprises a grip part, and wherein the nib is configured to release in the range of 0.1-3.0 μl upon a single physical topical application, wherein the physical topical application includes touching a topical area and keeping the nib in physical contact with the topical area in the range of 0.2-5 seconds.

11. The applicator according to claim 1, wherein the nib comprises a bundle of fibers with the adhesive material circumferentially surrounding the bundle over at least part of a bundle length, and wherein the nib comprises solvent welding connections between fibers.

12. The applicator according to claim 1, wherein a total amount of other components in the acidic liquid, other than the acid and the liquid, wherein the liquid is water, is less than 10 wt. %.

13. A kit of parts for topical application of an acidic liquid, the kit of parts comprising (a) a liquid release unit comprising a nib for a controlled release of the acidic liquid, and (b) a container unit comprising a container configured to contain the acidic liquid, wherein the container unit further comprises a liquid release element for application of the acidic liquid to the nib, wherein the nib comprises a plurality of fibers and an adhesive material adhering the fibers, wherein the nib comprises an elongated bundle of said fibres extending parallel to an axis of elongation, wherein the nib is configured for topical application of the acidic liquid, wherein the container comprises a polymeric container, and (i) wherein the polymeric container comprises a container wall comprising polypropylene and wherein the fibres comprise polypropylene comprising fibres, or (ii) wherein the polymeric container comprises a container wall comprising polyethylene terephthalate and wherein the fibres comprise polyethylene terephthalate comprising fibres, or (iii) wherein the polymeric container comprises a container wall comprising polytetrafluoroethylene and wherein the fibers comprise polytetrafluoroethylene comprising fibers, and wherein said container unit comprises said acidic liquid, wherein the acidic liquid has a pH less than 6, wherein the acidic liquid comprises an aqueous liquid, and wherein the acidic liquid comprises at least one or more of trichloro acetic acid and formic acid.

14. The kit of parts according to claim 13, wherein the liquid release unit comprises a tissue contact element selected from the group consisting of an adhesive superstrate, a wound bandage, a wound coating, and a patch, wherein the tissue contact element comprises said nib.

15. An acidic liquid for use in the treatment of a topical lesion selected from the group consisting of a superficial lesion, a skin lesion, a mucous membrane lesion, a nail lesion, a viral wart, verrucae, a water wart (molluscum contagiosum), a corn, a callus, an age spot, a solar lentigo, a senial lentigo, acne, keratosis pilaris, actinic keratosis, an ingrown toenails, onychomycosis, eyelid xanthelasma, psoriasis, a fungal nail infection, a skin tag, a mouth ulcer, a canker sore, a cold sore, a hemorrhoid, a genital wart, epidermodysplasia veruciformis, a HPV (human papillomavirus) caused skin disorder, scars, wrinkles, melisma, a tattoo, and a skin tag, wherein the acidic liquid is topically applied by contacting a topical area with the nib of an applicator according to claim 1, or the nib of the liquid release unit of the kit of parts comprising said liquid release unit and said container unit, wherein the acidic liquid has a pH less than 6, wherein the acidic liquid (30) comprises an aqueous liquid, and wherein the acidic liquid comprises at least one or more of trichloro acetic acid and formic acid.

16. The acidic liquid according to claim 15, for use in the treatment of a wart or a corn.
